(19) United States
(12) Patent Application Publication
(10) Pub. No.: US 2025/0381326 A1
(43) Pub. Date: Dec. 18, 2025

(54) BREAST SHIELD WITH SUPPORT

(71) Applicant: Zev Supplies Corp., Pompano Beach, FL (US)

(72) Inventors: Jianbiao Chen, Shantou City (CN); Jishun Huang, Shantou City (CN)

(21) Appl. No.: 19/238,255

(22) Filed: Jun. 13, 2025

(30) Foreign Application Priority Data
Jun. 13, 2024 (CN) 2024213436906

(51) Int. Cl. A61M 1/06

(52) U.S. Cl. CPC A61M 1/064; A61M 2205/0216

(57) ABSTRACT

The utility model discloses a breast shield with support brackets, belonging to the technical field of electric breast pumps. The exterior of the breast shield features several brackets of the support members, with the head portion of each support member embedded into the front end of the breast shield and the tail portion embedded into the rear end of the breast shield. The integration of the head portion and tail portion of the support members with the breast shield adopts an overmolding process, embedding the head portion and tail portion into the overmolding regions of the head portion and tail portion of the support member of the breast shield, respectively, forming an inseparable assembly with the breast shield. Compared with prior art, the breast shield offers superior structure and stability, and has lower manufacturing requirements and reduced costs. During use, it is not easy for users to experience separation, detachment, etc.

### RELATED APPLICATIONS

This application claims priority to pending Chinese Application No. 2024213436906, filed Jun. 13, 2024, the contents of which are incorporated by reference herein.

### Technical Field

This utility model patent relates to the technical field of electric breast pumps, particularly to a breast shield.

### Background Art

An electric breast pump employs a negative pressure device that acts on the human breast via a three-way suction device, stimulating milk secretion and extraction through the negative pressure. An electric breast pump typically comprises a main unit, a three-way component, a breast shield, and a feeding bottle. The said breast shield is made of soft liquid silicone.

CN103394135A discloses a breast shield structure of a breast pump, which comprises an inner breast shield assembly and an outer support frame. The inner breast shield assembly includes a large-port sealing inner ring sheet, a breast-protecting shield body, and a cup interface. These are seamlessly bonded into one piece via secondary injection molding. The said large port sealing inner ring sheet and the cup interface use PPSU material, while the breast shield body uses SIL liquid silicone. Prior art involves splitting the breast shield into multiple parts and using secondary injection molding or adhesive bonding and other processing techniques to achieve the integration of the supporting outer frame body with the multiple components of the breast shield.

After over a decade of application, although the above-mentioned prior art has excellent performance, it also exhibits the following shortcomings: complex material structure, high manufacturing requirements, and elevated costs.

CN 214911351 U discloses a breast shield support structure and a breast pump. The breast shield support structure comprises a breast shield and a support frame for supporting the breast shield. The breast shield comprises a suction segment and a connection segment. The outer wall of the suction segment is provided with the first groove, and the outer wall of the connection segment is provided with the second groove. The support frame comprises multiple connecting rods detachably connected at both ends to the respective grooves. The connecting rods (21) are made of rigid plastic. Its flaw lies in the detachable connection points between the connecting rods 21 and the outer wall of the suction segment or the groove of the outer wall of the connection segment, which can be easily disengaged with a little more force. Users may experience separation during daily use, especially during cleaning and sterilization of the breast shield, indicating inadequate stability and practicality.

Designing a breast shield with excellent stability, resistance to separation during use, simple manufacturing, and low cost presents a significant technical challenge.

### SUMMARY OF THE INVENTION

Addressing the deficiencies of prior art breast shields, this utility model is intended to provide a breast shield with support brackets. Compared with prior art, the breast shield offers superior structure and stability, and has lower manufacturing requirements and reduced costs. During use, it is not easy for users to experience separation, detachment, etc.

The utility model achieves the above performance through the following technical solutions: A breast shield with support members, wherein the breast shield is made of silicone and has a trumpet shape, with a large front trumpet-shaped opening for accommodating the breast and a rear end connected to a three-way connector. The exterior of the breast shield features several brackets of the support members. The head portion of each support member is embedded into the front end of the breast shield and encapsulated by the breast shield silicone; the tail portion is embedded into the rear end of the breast shield. Further, the integration of the head portion and tail portion of the support members with the breast shield adopts an overmolding process, embedding the head portion and tail portion into the overmolding regions of the head portion and tail portion of the support member of the breast shield, respectively, forming an inseparable assembly with the breast shield.

Further, the said overmolding process involves overmolding soft plastic onto hard plastic.

Further, more than one silicone sealing rings are arranged around the periphery of the rear end of the breast shield.

Further, the said support members are symmetrically distributed around the periphery of the breast shield.

Beneficial effect: Compared to prior art, the breast shield with support brackets said in the utility model has the following advantages:

The connection points between the support rod and the breast shield employ overmolding for robust bonding. The overmolding regions at these points are extensive. The head portion and tail portion of the support member are embedded into the overmolding regions of the breast shield to form an inseparable assembly with the breast shield. It cannot be easily disengaged with a little more force. The breast shield is a single silicone structural part. The support members are made of relatively rigid silicone with some elasticity. Repeated testing on the breast shield made by the utility model installed on breast pumps confirms that the breast shield fully meets the normal working requirements of breast pumps. At the same time, comparative tests against prior art connecting rods show no separation at the connection points between the support rod and the breast shield. The large overmolding regions at the connection joints between the head portion and tail portion of the support member and the breast shield ensure high structural stability of the breast shield with support brackets, preventing detachment during cleaning and sterilization.

### BRIEF DESCRIPTION OF THE DRAWINGS

FIG. 1 is a three-dimensional diagram of the breast shield of this utility model.

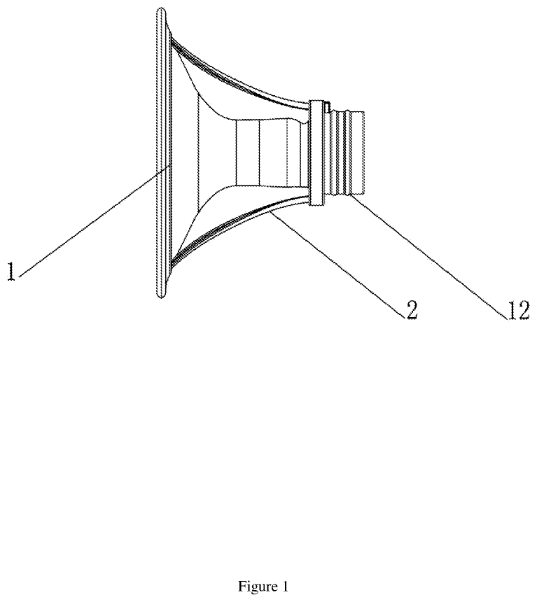

FIG. 2 is a schematic diagram of the breast shield without support brackets of this utility model.

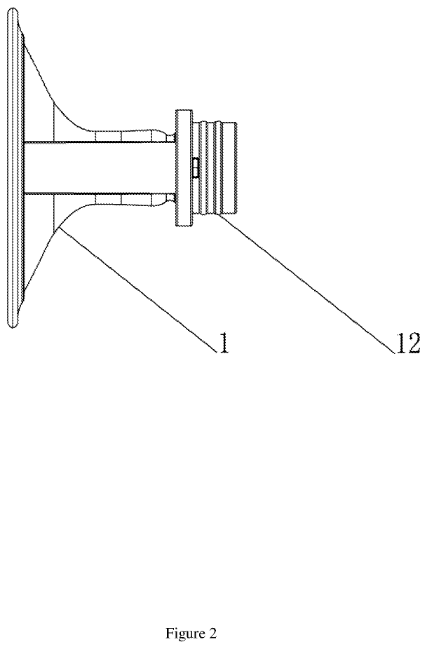

FIG. 3 is a cross-sectional schematic diagram of the breast shield of this utility model.

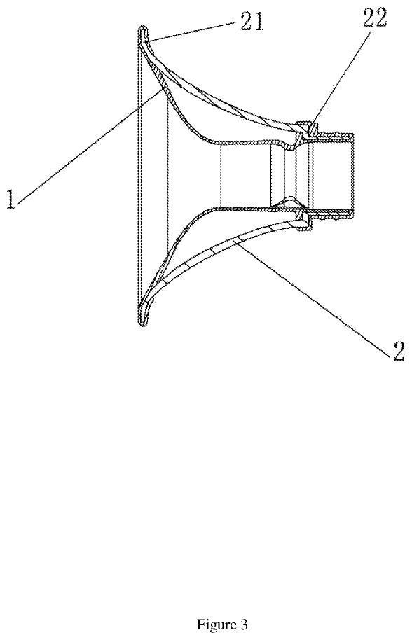

FIG. 4 is a schematic diagram of the overmolding region at the connection joints between the head portion of the support member and the breast shield.

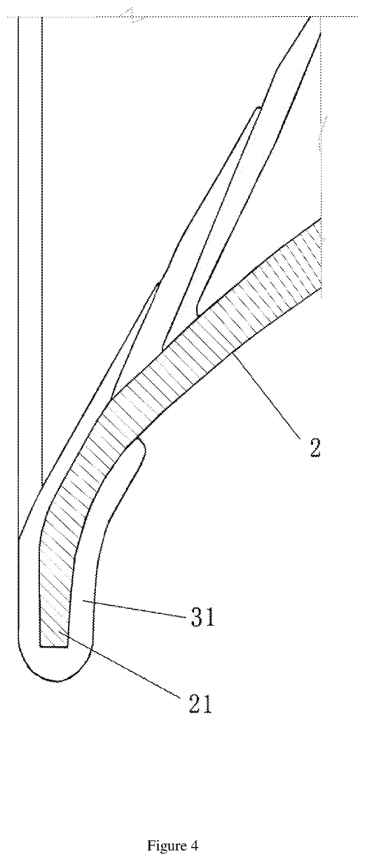

FIG. 5 is a schematic diagram of the overmolding region at the connection joints between the tail portion of the support member and the breast shield.

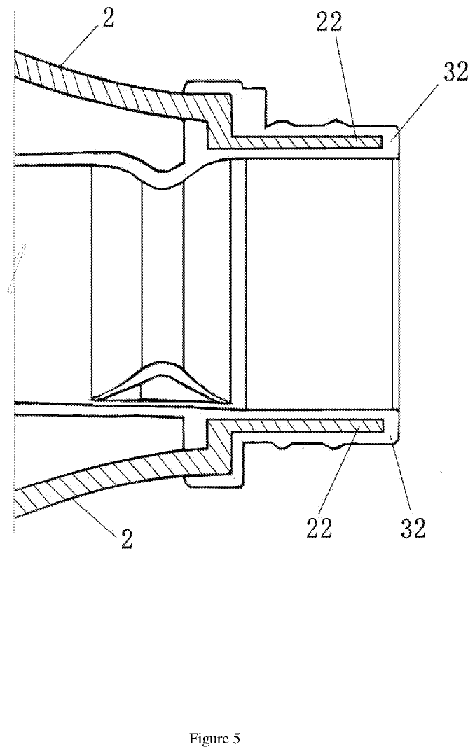

The meaning of the symbols in the drawings: 1—breast shield; 12—silicone sealing ring; 2—support member; 21—head portion of the support member; 22—tail portion of the support member; 31—overmolding region of head portion of the support member; 32—overmolding region of tail portion of the support member.

### EMBODIMENTS

The embodiments of this utility model are further described below with reference to the drawings of the specification.

A breast shield with support members, wherein the breast shield 1 is made of silicone and has a trumpet shape,
with a large front trumpet-shaped opening for accommodating the breast and a rear end connected to a three-way connector. The exterior of the breast shield 1 features several brackets of the support members 2.
   The head portion 21 of each support member is embedded into the front end of the breast shield 1 and encapsulated by the breast shield silicone; the tail portion 22 is embedded into the rear end of the breast shield 1. Further, the integration of the head portion 21 and tail portion 22 of the support members with the breast shield 1 adopts an overmolding process,
embedding the head portion 21 and tail portion 22 into the overmolding regions of the head portion 31 and tail portion 32 of the support member of the breast shield 1, respectively, forming an inseparable assembly with the breast shield 1.

Further, the said overmolding process involves overmolding soft plastic onto hard plastic.

Further, more than one silicone sealing rings 12 are arranged around the periphery of the rear end of the breast shield 1.

Further, the said support members 2 are symmetrically distributed around the periphery of the breast shield 1.

1. A breast shield with support members, wherein the breast shield (1) is made of silicone and has a trumpet shape, with a large front trumpet-shaped opening for accommodating the breast and a rear end connected to a three-way connector. The exterior of the breast shield (1) features several brackets of the support members (2). The head portion (21) of each support member is embedded into the front end of the breast shield (1), and the tail portion (22) is embedded into the rear end of the breast shield (1). The characteristic is that the integration of the head portion (21) and tail portion (22) of the support members with the breast shield (1) adopts an overmolding process, embedding the head portion (21) and tail portion (22) into the overmolding regions of the head portion (31) and tail portion (32) of the support member of the breast shield (1), respectively, forming an inseparable assembly with the breast shield (1).

2. The breast shield with support brackets as claimed in claim 1 is characterized in that the said overmolding process involves overmolding soft plastic onto hard plastic.

3. The breast shield with support brackets as claimed in claim 1 is characterized in that more than one silicone sealing rings (12) are arranged around the periphery of the rear end of the breast shield (1).

4. The breast shield with support brackets as claimed in claim 1, 2, or 3 is characterized in that the said support members (2) are symmetrically distributed around the periphery of the breast shield (1).
